(19) United States
(12) Patent Application Publication
(10) Pub. No.: US 2024/0001273 A1
(43) Pub. Date: Jan. 4, 2024

(54) FILTER DEVICE, PRODUCTION METHOD THEREFOR, AND AIR CONDITIONER

(71) Applicant: HONDA MOTOR CO., LTD., Tokyo (JP)

(72) Inventors: Naruhiko OKUBO, Tokyo (JP); Takaharu ECHIGO, Tokyo (JP); Mayu KURATA, Tokyo (JP); Yuta ISHII, Tokyo (JP); Yusuke HAYATSU, Tokyo (JP); Tatsuya NIWA, Tokyo (JP); Daisuke FUJII, Tokyo (JP); Takuya NAKATSURU, Tokyo (JP)

(21) Appl. No.: 18/253,595

(22) PCT Filed: Nov. 18, 2021
(86) PCT No.: PCT/JP2021/042436

§ 371 Date: May 19, 2023

(30) Foreign Application Priority Data
Nov. 20, 2020 (JP) 2020-193434

(51) Int. Cl. B01D 46/00; B01D 46/52; B01D 39/10; B01D 46/10

(52) U.S. Cl. CPC B01D 46/0028; B01D 46/0005; B01D 46/0001; B01D 46/522; B01D 39/10; B01D 46/10; B01D 2239/0442; B01D 2279/50; B01D 2275/10; B01D 2239/065

(57) ABSTRACT

A filter device 2 is provided with: a filter body 3 equipped with a filtering material 30; and a mesh-like filter cover 5 that is mounted to the filter body 3 in such a manner as to cover at least part of a filtering surface 32 of the filtering material 30. The filter cover 5 is provided with: a purification part 50 which includes multiple layers formed by folding a single sheet of metal mesh material and which has a rectangular shape that is substantially identical to that of the filtering surface 32 in a plan view; and a first leg 51 and a second leg 52 which intersect with the purification part 50 in a side view. The metal mesh material has a finely corrugated surface formed thereon. The entire outer peripheral edge 59 of the filter cover 5 is completely fitted within the filtering surface 32.

### TECHNICAL FIELD

The present invention relates to a filter device, a manufacturing method therefor, and an air conditioner. More specifically, the present invention relates to an air conditioner, a filter device provided in an air passage of the air conditioner, and a method for manufacturing the filter device.

### BACKGROUND ART

An automobile is equipped with an air conditioner that controls the temperature in the cabin to a comfortable temperature. For example, in the air passage of an automobile air conditioner shown in Patent Document 1, a filter material that collects dust included in air flowing through the air passage is provided in order to purify air introduced into the cabin.
Patent Document 1: Japanese Unexamined Patent Application, Publication No. H7-315044
Patent Document 2: Japanese Registered Utility Model No.

### DISCLOSURE OF THE INVENTION

### Problems to be Solved by the Invention

For the purpose of further enhancing the air purification function of an air conditioner, it is conceivable to cover the filter material for dust collection as described above with a mesh filter cover having air purification actions. Here, in order to ensure a sufficient purification function, it is necessary to employ a filter cover having a multi-layer structure with a plurality of stacked mesh members. In this case, in order to facilitate handling of the multi-layer mesh members, it is necessary to fix the outer edges of the plurality of mesh members with a frame member, for example, as in the invention shown in Patent Document 2. Therefore, there is a possibility that the filter device and the entire air conditioner may increase in weight and cost.

An object of the present invention is to provide a filter device provided with a multi-layer mesh material that is easy to handle, a manufacturing method therefor, and an air conditioner.

### Means for Solving the Problems

(1) A filter device according to the present invention includes: a filter material, and a mesh filter cover mounted to the filter material. The filter cover includes a multi-layer purification portion formed by folding a metal mesh material, and, on the metal mesh material, a minutely uneven surface is formed.

(2) In this case, it is preferable that the uneven surface has an antiviral action.

(3) In this case, it is preferable that the metal mesh material includes a central plane having a rectangular shape in plan view, and extension planes extending outward from four sides of the central plane in a developed state, and the filter cover is obtained by folding the metal mesh material on the four sides serving as fold lines.

(4) In this case, it is preferable that the metal mesh material includes a central plane having a rectangular shape in plan view and first and second extension planes extending outward from mutually facing first and second sides among four sides of the central plane, respectively, in a developed state, and the filter cover is obtained by folding the metal mesh material on the first and second sides serving as fold lines so that the central plane and the first and second extension planes are stacked.

(5) In this case, it is preferable that the metal mesh material includes third and fourth extension planes that extend outward from mutually facing third and fourth sides among the four sides in the developed state and are smaller than the first and second extension planes, and the filter cover is obtained by folding the metal mesh material on the third and fourth sides as fold lines.

(6) In this case, it is preferable that the metal mesh material includes a central plane having a rectangular shape in plan view, and an extension plane extending outward from one side of the central plane in a developed state, the filter cover includes the purification portion having a rectangular shape in plan view and including the central plane as one layer, and a leg portion intersecting with the purification portion and extending to the filter material side, the leg portion is formed by folding the extension plane on a fold line parallel to the one side, and a ridgeline of the leg portion on the filter material side is a mountain fold line when viewed from the filter material side.

(7) In this case, it is preferable that the filter device further includes a frame member having a rectangular shape in plan view and holding an outer edge of the filter material, and the leg portions are provided along an inner side of the frame member.

(8) In this case, it is preferable that the filter device further includes a frame member having a rectangular shape in plan view and holding an outer edge of the filter material, and the leg portions are provided along an outer side of the frame member.

A method for manufacturing a filter device according to the present invention includes: folding a metal mesh material; forming a minutely uneven surface on a surface of the metal mesh material that is folded, thereby manufacturing the filter cover; and mounting the filter cover to the filter material.

(10) An air conditioner according to the present invention includes: the filter device according to any one of (1) to (8); and an air passage through which air flows. The filter device is provided in the air passage, and the filter cover is provided on an air upstream side with respect to the filter material.

### Effects of the Invention

(1) A filter device according to the present invention includes a filter material, and a mesh filter cover mounted to the filter material. In the present invention, a metal mesh material having a minutely uneven surface is used as the filter cover. Here, the metal mesh material having the minutely uneven surface has air purification actions such as an antiviral action and an anti-mold action, for example, as described in Japanese Patent Application No. 2020-170093 by the applicant of the present application. Therefore, according to the filter device according to the present invention, it is possible to give, in addition to a dust collection action by the filter material, the purification actions by the filter cover.

When it is attempted to configure a multi-layer structured filter cover by stacking a plurality of metal mesh materials, a frame member for holding edge parts of the plurality of metal mesh materials is required in order to facilitate handling at the time of mounting the filter cover to the filter material. In contrast, in the present invention, a purification portion of the filter cover is formed by folding and multi-layering a metal mesh material. Thereby, according to the present invention, it is possible to hold the multi-layer structure of the purification portion of the filter cover by the metal mesh material itself and, therefore, facilitate handling at the time of mounting the filter cover to the filter material.

(2) In the filter device according to the present invention, it is possible to, by using the metal mesh material having the minutely uneven surface having the antiviral action, give the filter device the antiviral action by the filter cover, in addition to the dust collection action by the filter material.

(3) In the filter device according to the present invention, the metal mesh material includes a central plane having a rectangular shape in plan view, and extension planes extending outward from the four sides of the central plane in a developed state. Further, in the present invention, the metal mesh material folded on the four sides that demarcate the central plane from the extension planes around the central plane as fold lines is used as the filter cover. That is, in the present invention, the peripheral edge of the filter cover can be configured with fold lines of the metal mesh material. Therefore, according to the present invention, it is possible to hold the peripheral edge of the filter cover by the metal mesh material itself and, therefore, facilitate handling at the time of mounting the filter cover to the filter material.

(4) In the filter device according to the present invention, the metal mesh material includes the central plane having a rectangular shape in plan view, and first and second extension planes extending outward from mutually facing first and second sides among the four sides of the central plane, respectively, in a developed state. Further, in the present invention, the metal mesh material folded on the first and second sides serving as fold lines so that the central plane and the first and second extension planes are stacked is used as the filter cover. That is, in the present invention, the filter cover can be in a three-layer structure obtained by stacking the central plane and the first and second extension planes. Further, according to the present invention, at least two sides of the peripheral edge of the filter cover can be configured with fold lines of the metal mesh material. Therefore, according to the present invention, it is possible to hold the peripheral edge by the metal mesh material itself and, therefore, facilitate handling at the time of mounting the filter cover to the filter material.

(5) In the filter device according to the present invention, the metal mesh material includes third and fourth extension planes that extend outward from mutually facing third and fourth sides among the four sides of the central plane in the developed state and are smaller than the first and second extension planes. Further, in the present invention, the metal mesh material folded on the third and fourth sides serving as fold lines is used as the filter cover. Therefore, according to the present invention, it is possible to achieve the filter cover with a three-layer structure as described above and configure the entire peripheral edge with fold lines. Therefore, according to the present invention, it is possible to hold the peripheral edge by the metal mesh material itself and, therefore, facilitate handling at the time of mounting the filter cover to the filter material.

(6) In the filter device according to the present invention, the filter cover is provided with the purification portion having a rectangular shape in plan view and including the central plane as one layer, and leg portions intersecting with the purification portion and extending to the filter material side. According to the present invention, it is possible to mount the filter cover to the filter material by engaging the leg portions with the filter material. Further, in the present invention, the leg portions described above are formed by folding the extension planes of the metal mesh material, and ridgelines of the leg portions on the filter material side are mountain fold lines when viewed from the filter material side. Thereby, the leg portions of the filter cover can be strengthened with a simple configuration.

(7) In the filter device according to the present invention, the leg portions of the filter cover are provided along the inner side of the rectangular frame member that holds the outer edge of the filter material. Thereby, it is possible to mount the filter cover to the filter material by simply engaging the leg portions with the inner side of the frame member for holding the filter material.

(8) In the filter device according to the present invention, the leg portions of the filter cover are provided along the outer side of the rectangular frame member that holds the outer edge of the filter material. Thereby, it is possible to mount the filter cover to the filter material by simply engaging the leg portions with the outer side of the frame member for holding the filter material.

(9) There is a possibility that the minutely uneven surface formed on the surface of the metal mesh material is damaged by being bent or folded, and the purification actions thereof deteriorates. To address this, in the method for manufacturing a filter device according to the present embodiment, a filter cover is manufactured by, after folding a metal mesh material, forming a minutely uneven surface on the surface of the metal mesh material. Thereby, it is possible to manufacture the filter cover with a multi-layer structure without damaging the uneven surface.

(10) An air conditioner according to the present invention is provided with an air passage through which air flows, and the filter device provided in the air passage. Further, in the present invention, the filter cover of the filter device is provided on an air upstream side with respect to the filter material. Thereby, it is possible to introduce air that has been purified by passing through the filter cover, into the filter material.

### BRIEF DESCRIPTION OF THE DRAWINGS

FIG. 1 is a sectional view showing a configuration of an air conditioner according to a first embodiment of the present invention;

FIG. 2 is a perspective view of a filter device viewed from an air upstream side;

FIG. 3 is a sectional view of the filter device along a line III-III in FIG. 2;

FIG. 4 is a sectional view of the filter device along a line IV-IV in FIG. 2;

FIG. 5 is a development view of a metal mesh material;

FIG. 6 is a flowchart showing a procedure for a method for manufacturing the filter device;

FIG. 7 is a diagram for illustrating a procedure for folding the metal mesh material (1);

FIG. 8 is a diagram for illustrating the procedure for folding the metal mesh material (2);

FIG. 9 is a diagram for illustrating the procedure for folding the metal mesh material (3);

FIG. 10 is a sectional view of a filter device according to a second embodiment of the present invention;

FIG. 11 is a sectional view along a line including first and second leg portions of the filter device;

FIG. 12 is a sectional view along a line including first and second edge parts of the filter device;

FIG. 13 is a diagram for illustrating the procedure for folding a metal mesh material (1); and

FIG. 14 is a diagram for illustrating the procedure for folding the metal mesh material (2).

### PREFERRED MODE FOR CARRYING OUT THE INVENTION

### First Embodiment

Configurations of a filter device and an air conditioner equipped with the filter device, and a method for manufacturing the filter device according to a first embodiment of the present invention will be described below with reference to the drawings.

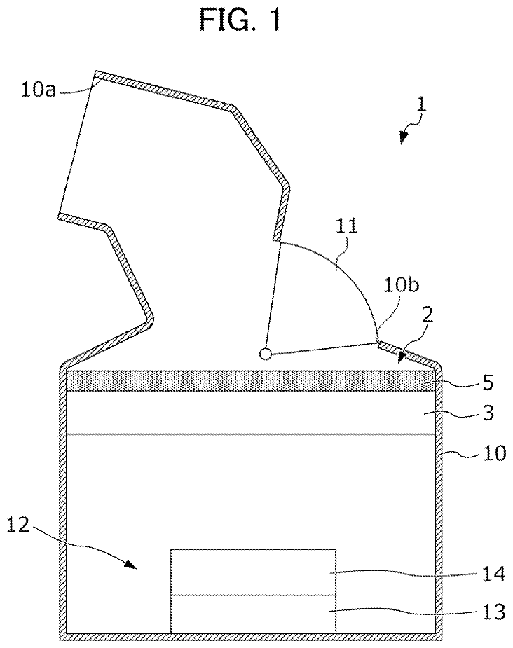

FIG. 1 is a sectional view showing a configuration of an air conditioner 1 according to the present embodiment. The air conditioner 1 is provided with an air passage 10 which extends in the vertical direction and in which air flows, and an intake damper 11, a filter device 2, and a blower 12 that are provided in the air passage 10 from the air upstream side (the upper side in FIG. 1) to the air downstream side (the lower side in FIG. 1) in that order. The air conditioner 1 is accommodated, for example, inside an instrument panel in front of the passenger seat of a vehicle.

At the end part of the air passage 10 on the upstream side, an outside air inlet 10a that introduces air outside the cabin into the inside of the air passage 10 and an inside air inlet 10b that introduces air in the cabin into the inside of the air passage 10 are provided. The outside air and inside air inlets 10a and 10b are selectively opened to the inside of the air passage 10 by the intake damper 11. On the air downstream side of the filter device 2 in the air passage 10, the blower 12, and an evaporator, a heater core, and the like that are not shown are provided. FIG. 1 shows a state in which the outside air inlet 10a is opened to introduce air outside the cabin, by tilting the intake damper 11 to the inside air inlet 10b side.

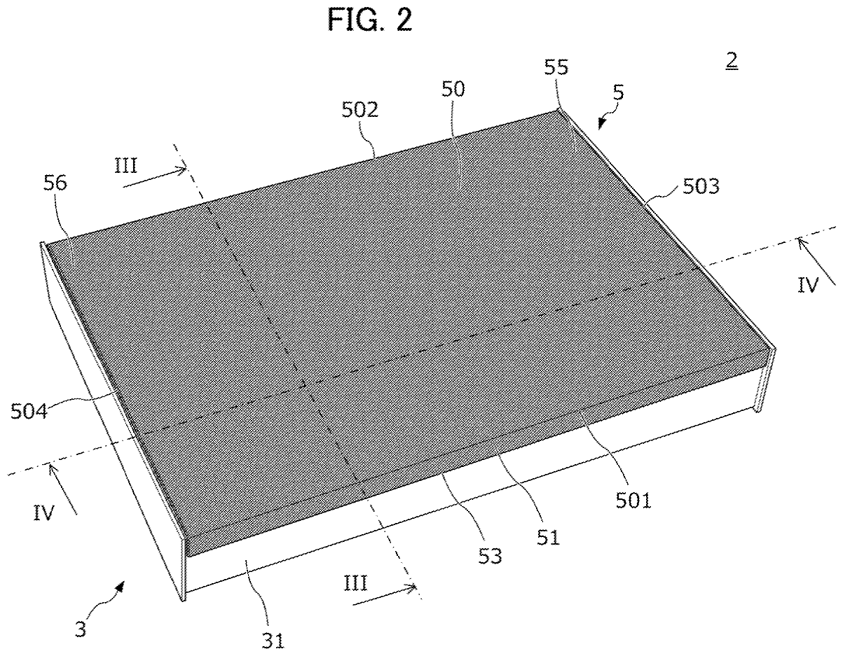

The filter device 2 purifies air flowing through the air passage 10. As described below in detail with reference to FIGS. 2 to 9, the filter device 2 is provided with a filter body 3 having a rectangular box shape, and a filter cover 5 formed by folding one metal mesh material. In the air passage 10 extending in the vertical direction, the filter device 2 is installed substantially orthogonal to the air passage 10, that is, being substantially horizontal. In the present embodiment, description will be made on a case where the filter device 2 is installed in the air passage 10 such that the filter cover 5 is provided on the air upstream side with respect on the filter body 3 in the air passage 10, as shown in FIG. 1. The present invention, however, is not limited thereto. The filter device 2 may be installed in the air passage 10 such that the filter cover 5 is provided on the filter body 3 on the air downstream side in the air passage 10.

As described later, the filter device 2 has a dust collection action of collecting dust included in air and an antiviral action of collecting droplets including viruses in the air and inactivating the viruses. Therefore, the filter device 2 provided in the air passage 10 purifies air flowing through the air passage 10 by the dust collection action and the antiviral action.

The blower 12 forms a flow of air from the outside air inlet 10a side or the inside air inlet 10b side toward the filter device 2 side in the air passage 10, by causing a fan 14 to rotate by a motor 13. Therefore, air introduced from the outside air inlet 10a or the inside air inlet 10b by causing the blower 12 to operate is introduced, after being purified by the dust collection action and antiviral action of the filter device 2, into the cabin through a communication port not shown, which is provided at a lower part of the air passage 10, via the evaporator, the heater core and the like.

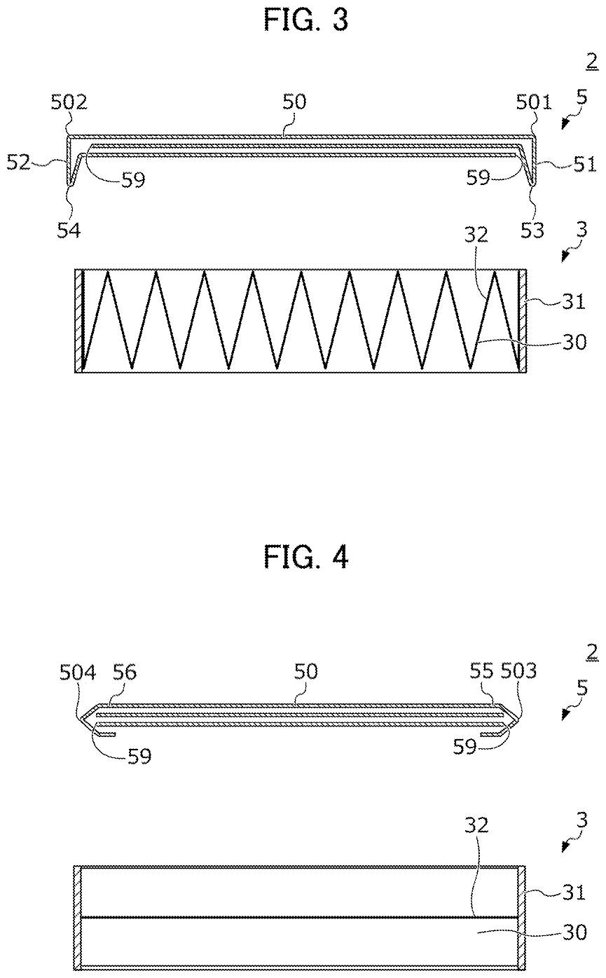

FIG. 2 is a perspective view of the filter device 2 viewed from the air upstream side. FIG. 3 is a sectional view of the filter device 2 along a line III-III in FIG. 2. FIG. 4 is a sectional view of the filter device 2 along a line IV-IV in FIG. 2.

The filter device 2 is provided with the filter body 3 having a rectangular box shape, and the mesh filter cover 5 mounted to the filter body 3, to cover at least a part of a filter surface 32 of the filter body 3 on the air upstream side. In the present embodiment, description will be made on a case where the filter cover 5 is mounted to the filter body 3 to cover the entire surface of the filter surface 32 of the filter body 3. The present invention, however, is not limited thereto. It is sufficient if at least a part of the filter surface 32 is covered with the filter cover 5.

The filter body 3 is provided with a porous filter material 30 that collects dust included in air, and a frame member 31 that holds the outer edge of the filter material 30. The filter material 30 has a rectangular shape in plan view. The surface of the filter material 30 on the air upstream side is the filter surface 32 into which air flows. Though description will be made below on a case where a nonwoven fabric folded in a bellows shape is used as the filter material 30 as shown in FIG. 3, the present invention is not limited thereto. The frame member 31 has a cylindrical shape extending in the thickness direction of the filter material 30, and has a rectangular shape that is slightly larger than the filter material 30 in plan view. The outer edge of the filter material 30 is fixed to the inner surface of the frame member 31.

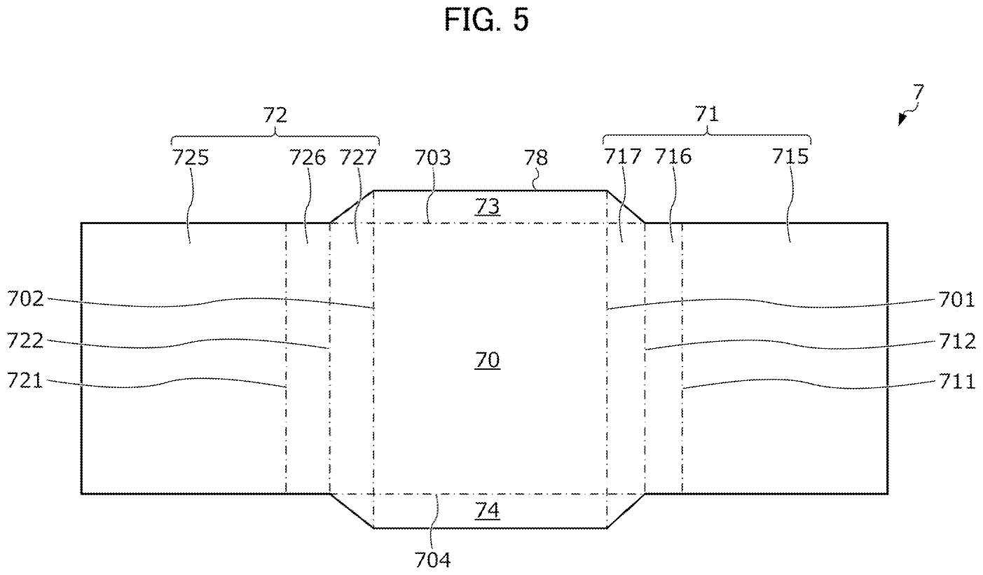

The filter cover 5 is formed by folding a belt-shaped metal mesh material 7 in a developed state as shown in FIG. 5 on a plurality of lines 701, 702, 703, 704, 711, 712, 721, and 722 indicated by one-dot-chain lines in FIG. 5 serving as fold lines, and has substantially the same rectangular shape as the filter material 30 in plan view. As the metal mesh material 7 constituting the filter cover 5, a material with a minutely uneven surface having an antiviral action formed on its surface is used. Although a case will be described below in which an aluminum mesh material, i.e., what is obtained by plainly weaving aluminum fibers is used as the metal mesh material 7, the present invention is not limited thereto. Therefore, the filter device 2 has the dust collection action of collecting dust included in air and the antiviral action of collecting droplets including viruses in the air and inactivating the viruses.

In plan view, the filter cover 5 is provided with a purification portion 50 having substantially the same rectangular shape as the filter surface 32, belt-shaped first and second leg portions 51 and 52 extending along mutually facing first and second sides 501 and 502 among the four sides of the purification portion 50.

In a state of the filter cover 5 being mounted to the filter body 3, the purification portion 50 covers the entire surface of the filter surface 32. As shown in FIG. 3, the purification portion 50 has a three-layer structure formed by folding the metal mesh material 7 so that a central plane 70, and first and second lower layer planes 715 and 725 which are parts of the metal mesh material 7 (see FIG. 5) are stacked.

As shown in FIG. 4, a first edge part 55 of the purification portion 50 on a third side 503 side has a four-layer structure, the third side 503 being orthogonal to the first and second sides 501 and 502, and the four-layer structure being formed by folding the metal mesh material 7 so that the central plane 70, the first and second lower layer planes 715 and 725, and a first ear plane 73 which are parts of the metal mesh material 7 (see FIG. 5) are stacked. Further, a second edge part 56 of the purification portion 50 on a fourth side 504 side has a four-layer structure, the fourth side 504 facing the third side 503, and the four-layer structure being formed by folding the metal mesh material 7 so that the central plane 70, the first and second lower layer planes 715 and 725, and a second ear plane 74 which are parts of the metal mesh material 7 (see FIG. 5) are stacked.

The first and second leg portions 51 and 52 are substantially parallel to each other in side view. The first and second leg portions 51 and 52 intersect with the purification portion 50 and extend to the filter body 3 side. As shown in FIG. 3, each of the first and second leg portions 51 and 52 has a two-layer structure formed by folding the metal mesh material 7. Further, first and second ridgelines 53 and 54 of the first and second leg portions 51 and 52 on the filter body 3 side are mountain fold lines when viewed from the filter body 3 side.

The filter cover 5 configured as described above is provided on the filter body 3 such that the purification portion 50 covers the filter surface 32, and the first and second leg portions 51 and 52 are along the outer side of the frame member 31. In the present embodiment, description will be made on the case where the first and second leg portions 51 and 52 are provided along the outer side of the frame member 31. The present invention, however, is not limited thereto. The first and second leg portions 51 and 52 may be provided along the inner side of the frame member 31.

In the filter cover 5 formed by folding the one metal mesh material 7 as described above, an outer edge 59 of the metal mesh material 7 is entirely included in the purification portion 50 in plan view. That is, in the state of the filter cover 5 being mounted to the filter body 3, the entire outer edge 59 of the filter cover 5 is included within the filter surface 32 in plan view.

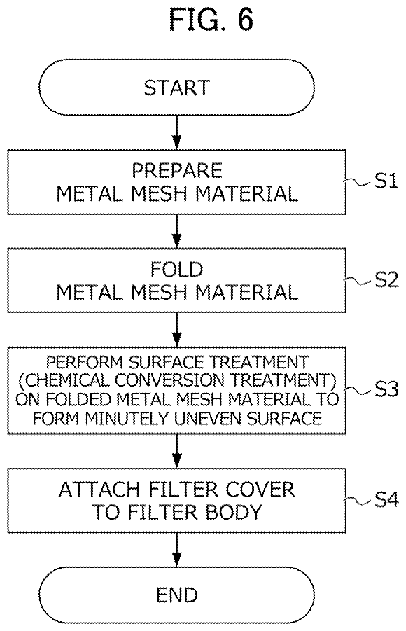

FIG. 6 is a flowchart showing a procedure for a method for manufacturing the filter device 2 as described above. Though description will be made below on the case of manufacturing the filter device 2 with a worker as a subject, the present invention is not limited thereto. The filter device 2 may be manufactured with a machine as a subject instead of the worker.

At S1, the worker prepares the belt-shaped metal mesh material 7 as shown in FIG. 5.

As shown in the development view of FIG. 5, the metal mesh material 7 in the developed state has a long belt shape extending in left and right directions in FIG. 5.

The metal mesh material 7 in the developed state includes the central plane 70 having substantially the same rectangular shape as the filter surface 32, a first extension plane 71 extending outward from a first side 701 of the central plane 70, a second extension plane 72 extending outward from a second side 702 facing the first side 701 of the central plane 70, the first ear plane 73 extending outward from the third side 703 orthogonal to the first and second sides 701 and 702 of the central plane 70, the second ear plane 74 extending outward from the fourth side 704 facing the third side 703 of the central plane 70 in plan view.

The first extension plane 71 is demarcated into the first lower layer plane 715, a first inner side leg plane 716, and a first outer side leg plane 717 from outside to the central plane 70 side in that order by the two lines 711 and 712 extending parallel to the first side 701. The first lower layer plane 715 has a rectangular shape in plan view, and constitutes a part of the purification portion 50 of the filter cover 5 in a folded state. The length of the first lower layer plane 715 in the extension direction of the third side 703 is slightly shorter than the length of the central plane 70 in the extension direction of the third side 703. The first inner side leg plane 716 has a belt shape extending along the first side 701 in plan view, and constitutes a part of the first leg portion 51 of the filter cover 5 in the folded state. The first outer side leg plane 717 has substantially the same belt shape as the first inner side leg plane 716 in plan view, and constitutes a part of the first leg portion 51 of the filter cover 5 in the folded state. The first lower layer plane 715 and the first inner side leg plane 716 are demarcated by the line 711. The first inner side leg plane 716 and the first outer side leg plane 717 are demarcated by the line 712. The line 712 constitutes the first ridgeline 53 of the filter cover 5 in the folded state.

The second extension plane 72 is demarcated into the second lower layer plane 725, a second inner side leg plane 726, and a second outer side leg plane 727 from outside to the central plane 70 side in that order by the two lines 721 and 722 extending parallel to the second side 702. The second lower layer plane 725 has a rectangular shape in plan view, and constitutes a part of the purification portion 50 of the filter cover 5 in the folded state. The length of the second lower layer plane 725 in the extension direction of the third side 703 is slightly shorter than the length of the central plane 70 in the extension direction of the third side 703. The second inner side leg plane 726 has a belt shape extending along the second side 702 in plan view, and constitutes a part of the second leg portion 52 of the filter cover 5 in the folded state. The second outer side leg plane 727 has substantially the same belt shape as the second inner side leg plane 726 in plan view, and constitutes a part of the second leg portion 52 of the filter cover 5 in the folded state. The second lower layer plane 725 and the second inner side leg plane 726 are demarcated by the line 721. The second inner side leg plane 726 and the second outer side leg plane 727 are demarcated by the line 722. The line 722 constitutes the second ridgeline 54 of the filter cover 5 in the folded state.

The first ear plane 73 has a belt shape extending along the third side 703 in plan view and is smaller than the first and second extension planes 71 and 72. The first ear plane 73 constitutes a part of the first edge part 55 of the filter cover 5 in the folded state. The second ear plane 74 has a belt shape extending along the fourth side 704 in plan view and is smaller than the first and second extension plane 71 and 72. The second ear plane 74 constitutes the second edge part 56 of the filter cover 5 in the folded state.

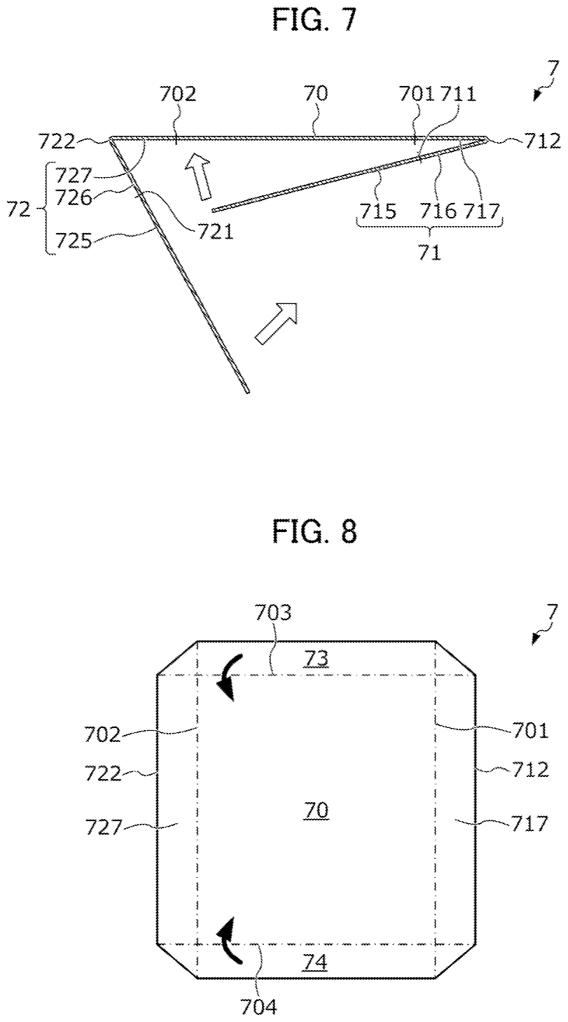

Returning to FIG. 6, at S2, the worker folds the metal mesh material 7 in accordance with a procedure shown in FIGS. 7, 8, and 9, with the plurality of lines 701, 702, 703, 704, 711, 712, 721, and 722 indicated by the one-dot-chain lines in FIG. 5 as fold lines.

Each of FIGS. 7, 8, and 9 is a diagram for illustrating the procedure for folding the metal mesh material 7.

First, as shown in FIG. 7, after folding the first extension plane 71 along the line 712 so that the central plane 70 and the first lower layer plane 715 are stacked, the worker further folds the second extension plane 72 along the line 722 so that the central plane 70 and the first and second lower layer planes 715 and 725 are stacked.

Next, as shown in FIG. 8, after folding the first ear plane 73 along the third side 703, the worker further folds the second ear plane 74 along the fourth side 704. At this time, it is desirable to mountain-fold the third and fourth sides 703 and 704 when they are viewed from the central plane 70 side so that the first and second ear planes 73 and 74 are stacked on the second lower layer plane 725. Thereby, parts of the filter cover 5 to be the first and second edge parts 55 and 56 are formed.

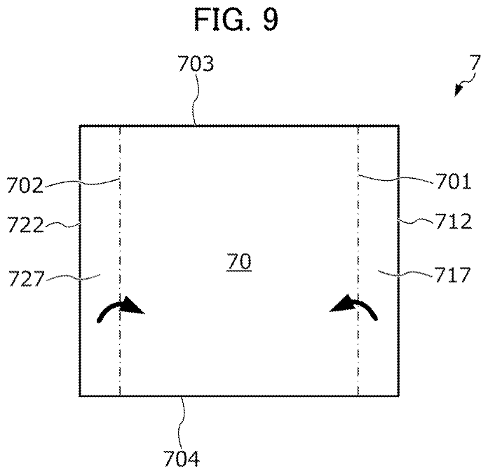

Next, as shown in FIG. 9, after folding the first outer side leg plane 717 and the first inner side leg plane 716 along the first side 701, the worker further folds the second outer side leg plane 727 and the second inner side leg plane 726 along the second side 702. At this time, it is desirable to mountain-fold the first and second sides 701 and 702 when they are viewed from the central plane 70 side so that the first and second outer side leg plane 717 and 727 are substantially perpendicular to the central plane 70. Thereby, parts of the filter cover 5 to be the purification portion 50 and the first and second leg portions 51 and 52 are formed.

By folding the metal mesh material 7 by the procedure as above, it is possible to include the entirety of an outer edge 78, which is a part to become the outer edge 59 of the filter cover 5, within the central plane 70 in plan view.

Returning to FIG. 6, at S3, the worker forms a minutely uneven surface having an antiviral action on the surface of the metal mesh material 7, by performing predetermined surface treatment (for example, chemical conversion treatment) on the folded metal mesh material 7, to manufacture the filter cover 5. More specifically, by performing zinc phosphate conversion treatment directly on the metal mesh material 7 folded at S2, minute unevenness is formed on the surface of the metal mesh material 7. Especially, by setting the interval between protrusions within a range of 0.5 [μm] to 1.5 [μm], and further setting the area of each recess within a range of 0.05 [μm²] to 2.0 [μm²], it is possible to form the minute uneven surface having the antiviral action on the surface of the metal mesh material 7. A specific procedure for the chemical conversion treatment for forming such a minutely uneven surface having an antiviral action on the surface of the metal mesh material 7 is described, for example, in Japanese Patent Application No. 2020-170093 by the applicant of the present application, and, therefore, detailed description will be omitted here.

At S4, by engaging the first and second leg portions 51 and 52 of the filter cover 5 with the frame member 31 of the filter body 3, the worker mounts the filter cover 5 to the filter body 3. Thereby, the filter device 2 has been manufactured.

According to the present embodiment, the following effects are obtained.

(1) The filter device 2 is provided with the filter material 30, and the mesh filter cover 5 mounted to the filter material 30 to cover at least a part of the filter surface 32 of the filter material 30. In the filter device 2, the metal mesh material 7 having a minutely uneven surface is used as the filter cover 5. Here, the metal mesh material 7 having the minutely uneven surface has air purification actions such as the antiviral action and the anti-mold action as described above. Therefore, according to the filter device 2, it is possible to give, in addition to the dust collection action by the filter material 30, the purification actions by the filter cover 5. Further, in the filter device 2, the purification portion 50 of the filter cover 5 is formed by folding and multi-layering the metal mesh material 7. Thereby, according to the filter device 2, it is possible to hold the multi-layer structure of the purification portion 50 of the filter cover 5 by the metal mesh material 7 itself and, therefore, facilitate handling at the time of mounting the filter cover 5 to the filter material 30.

(2) In the filter device 2, it is possible to, by using the metal mesh material 7 having the minutely uneven surface having the antiviral action, give the filter device 2 the antiviral action by the filter cover 5, in addition to the dust collection action by the filter material 30.

(3) In the filter device 2, the metal mesh material 7 includes the central plane 70 having a rectangular shape in plan view, and the extension planes 71 and 72 and the ear planes 73 and 74 extending outward from the four sides 701 to 704 of the central plane 70 in a developed state. Further, in the filter device 2, the metal mesh material 7 folded on the four sides 701 to 704 that demarcate the central plane 70 from the extension planes 71 and 72 and the ear planes 73 and 74 around the central plane 70 as fold lines, is used as the filter cover 5. That is, in the filter device 2, the peripheral edge of the filter cover 5 can be configured with fold lines of the metal mesh material 7. Therefore, according to the filter device 2, it is possible to hold the peripheral edge of the filter cover 5 by the metal mesh material 7 itself and, therefore, facilitate handling at the time of mounting the filter cover 5 to the filter material 30.

(4) In the filter device 2, the metal mesh material 7 includes the central plane 70 having a rectangular shape in plan view, and the first and second extension planes 71 and 72 extending outward from the mutually facing first and second sides 701 and 702 among the four sides 701 to 704 of the central plane 70, respectively, in a developed state. Further, in the filter device 2, the metal mesh material 7 folded on the first and second sides 701 and 702 as fold lines so that the central plane 70 and the first and second extension planes 71 and 72 are stacked is used as the filter cover 5. That is, in the filter device 2, the filter cover 5 can be in a three-layer structure obtained by stacking the central plane 70 and the first and second lower layer planes 715 and 725. Further, according to the filter device 2, at least two sides of the peripheral edge of the filter cover 5 can be configured with fold lines of the metal mesh material 7. Therefore, according to the filter device 2, it is possible to hold the peripheral edge by the metal mesh material 7 itself and, therefore, facilitate handling at the time of mounting the filter cover 5 to the filter material 30.

(5) In the filter device 2, the metal mesh material 7 includes the first and second ear planes 73 and 74 that extend outward from the mutually facing third and fourth sides 703 and 704 among the four sides 701 to 704 of the central plane 70 in the developed state and are smaller than the first and second extension planes 71 and 72. Further, in the filter device 2, the metal mesh material 7 folded on the third and fourth sides 703 and 704 serving as fold lines is used as the filter cover 5. Therefore, according to the filter device 2, it is possible to cause the purification portion 50 of the filter cover 5 to be in a three-layer structure as described above and configure all the four sides 501 to 504 of the peripheral edge thereof with fold lines. Therefore, according to the filter device 2, it is possible to hold the four sides 501 to 504 by the metal mesh material 7 itself and, therefore, facilitate handling at the time of mounting the filter cover 5 to the filter material 30.

(6) In the filter device 2, the filter cover 5 is provided with the purification portion 50 having a rectangular shape in plan view and including the central plane 70 as one layer, and the first and second leg portions 51 and 52 intersecting with the purification portion 50 and extending to the filter body 3 side. According to the filter device 2, it is possible to mount the filter cover 5 to the filter body 3 by engaging the first and second leg portions 51 and 52 with the filter body 3. Further, in the filter device 2, the first and second leg portions 51 and 52 as described above are formed by folding the first and second extension planes 71 and 72 of the metal mesh material 7, and the first and second ridgelines 53 and 54 of the first and second leg portions 51 and 52 on the filter body 3 side are mountain fold lines when viewed from the filter body 3 side. Thereby, the first and second leg portions 51 and 52 of the filter cover 5 can be strengthened with a simple configuration.

(7) In the filter device 2, the first and second leg portions 51 and 52 of the filter cover 5 are provided along the inner side of the rectangular frame member 31 that holds the outer edge of the filter material 30. Thereby, it is possible to mount the filter cover 5 to the filter material 30 by simply engaging the first and second leg portions 51 and 52 with the inner side of the frame member 31 for holding the filter material 30.

(8) In the filter device 2, the first and second leg portions 51 and 52 of the filter cover 5 are provided along the outer side of the frame member 31. Thereby, it is possible to mount the filter cover 5 to the filter material 30 by simply engaging the first and second leg portions 51 and 52 with the outer side of the frame member 31 for holding the filter material 30.

(9) In the method for manufacturing the filter device 2 according to the present embodiment, the filter cover 5 is manufactured by, after folding the metal mesh material 7, forming a minutely uneven surface on the surface of the metal mesh material 7. Thereby, it is possible to manufacture the filter cover 5 having a multi-layer structure without damaging the uneven surface.

(10) The air conditioner 1 according to the present embodiment is provided with the air passage 10 through which air flows, and the filter device 2 provided in the air passage 10. Further, in the air conditioner 1, the filter cover 5 of the filter device 2 is provided on the air upstream side with respect to the filter body 3. Thereby, it is possible to introduce air that is purified by passing through the filter cover 5, into the filter material 30.

### Second Embodiment

Next, a configuration of a filter device according to a second embodiment of the present invention will be described with reference to drawings. In the description below, the same components as those of the filter device 2 according to the first embodiment will be given the same reference numerals, and detailed description will be omitted. The filter device according to the present embodiment is different from the filter device 2 according to the first embodiment mainly in the configuration of a filter cover.

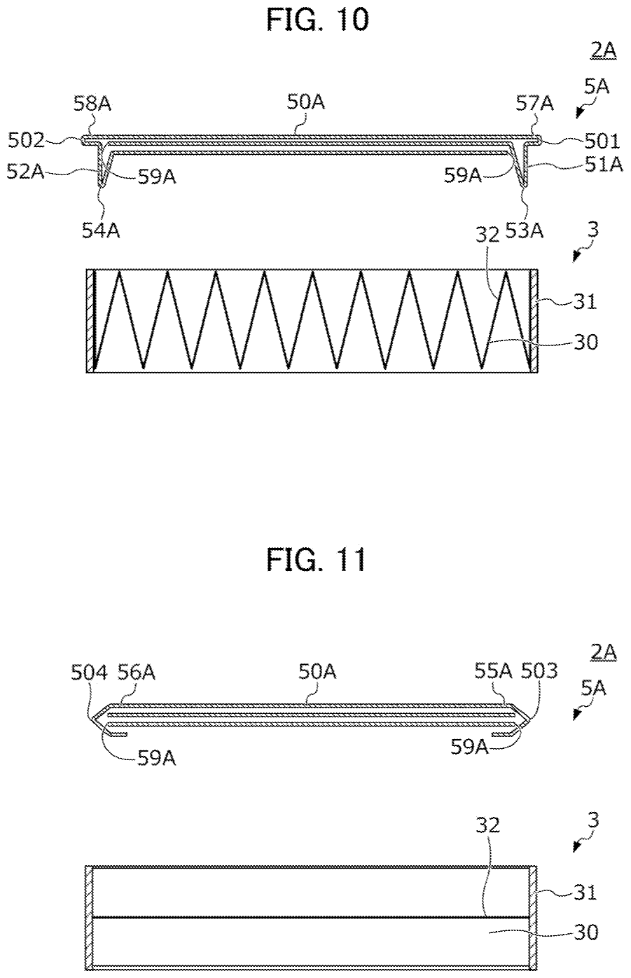

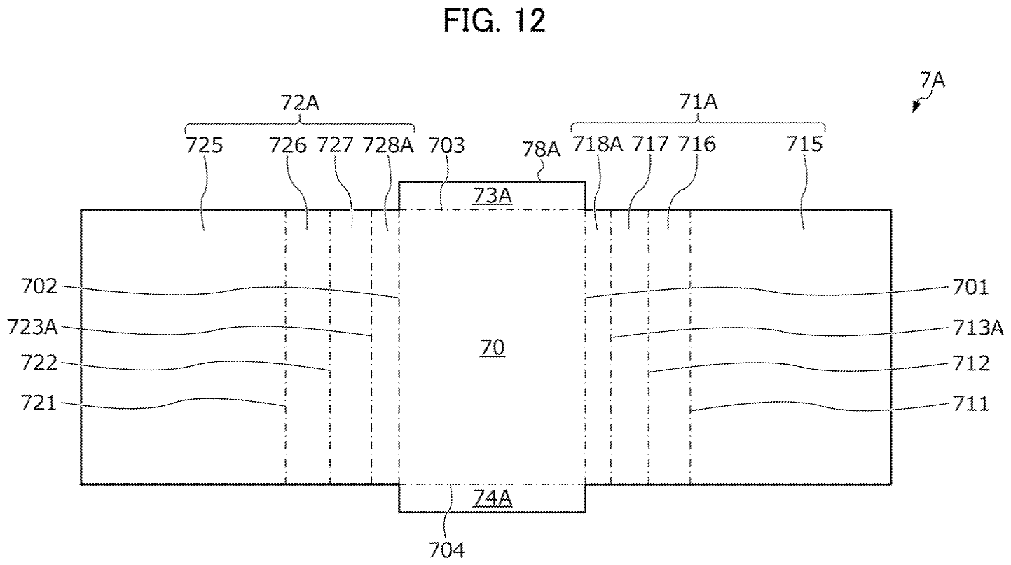

FIG. 10 is a sectional view along a line including first and second leg portions 51A and 52A of a filter device 2A according to the present embodiment. FIG. 11 is a sectional view of the filter device 2A along a line orthogonal to FIG. 10. More specifically, FIG. 11 is a sectional view along a line including first and second edge parts 55A and 56A of the filter device 2A. FIG. 12 is a development view of a metal mesh material 7A used for a filter cover 5A of the filter device 2A.

The filter cover 5A according to the present embodiment is formed by folding one metal mesh material 7A in a long belt shape as shown in FIG. 12 in a developed state, on a plurality of lines 701, 702, 703, 704, 711, 712, 713A, 721, 722, and 723A indicated by one-dot-chain lines in FIG. 12 and serving as fold lines. As the metal mesh material 7A constituting the filter cover 5A, a material with a minutely uneven surface having an antiviral action formed on its surface is used similarly to the first embodiment.

In plan view, the filter cover 5A is provided with a purification portion 50A having substantially the same rectangular shape as a filter surface 32, the belt-shaped first and second leg portions 51A and 52A extending along mutually facing first and second sides 501 and 502 among the four sides of the purification portion 50A.

In a state of the filter cover 5A being mounted to the filter body 3, the purification portion 50A covers the entire surface of the filter surface 32. As shown in FIG. 10, the purification portion 50A has a three-layer structure formed by folding the metal mesh material 7A so that a central plane 70, and first and second lower layer planes 715 and 725 which are parts of the metal mesh material 7 (see FIG. 12) are stacked.

As shown in FIG. 11, a first edge part 55A of the purification portion 50A on a third side 503 side has a four-layer structure formed by folding the metal mesh material 7A so that the central plane 70, the first and second lower layer planes 715 and 725, and a first ear plane 73A which are parts of the metal mesh material 7A (see FIG. 12) are stacked. Further, a second edge part 56A of the purification portion 50A on a fourth side 504 side has a four-layer structure formed by folding the metal mesh material 7A so that the central plane 70, the first and second lower layer planes 715 and 725, and a second ear plane 74A which are parts of the metal mesh material 7A (see FIG. 12) are stacked.

As shown in FIG. 10, a first overhang portion 57A, which is an edge part of the purification portion 50A on the first side 501 side, has a two-layer structure formed by folding the metal mesh material 7A so that the central plane 70 and a first overhang plane 718A which are parts of the metal mesh material 7A (see FIG. 12) are stacked. Further, a second overhang portion 58A, which is an edge part of the purification portion 50A on the second side 502 side, has a two-layer structure formed by folding the metal mesh material 7A so that the central plane 70 and a second overhang plane 728A which are parts of the metal mesh material 7A (see FIG. 12) are stacked.

The first and second leg portions 51A and 52A are substantially parallel to each other in side view. The first and second leg portions 51A and 52A intersect with the purification portion 50A and extend to the filter body 3 side from slightly inner side of the first and second sides 501 and 502. As shown in FIG. 10, each of the first and second leg portions 51A and 52A has a two-layer structure formed by folding the metal mesh material 7A. First and second ridgelines 53A and 54A of the first and second leg portions 51A and 52A on the filter body 3 side are mountain fold lines when viewed from the filter body 3 side.

In the filter cover 5A configured as above, the purification portion 50A covers the filter surface 32; the first and second overhang portions 57A and 58A of the purification portion 50A are engaged with the frame member 31 of the filter body 3; and the first and second leg portions 51A and 52A are provided on the filter body 3 along the inner side of the frame member 31.

Further, as described above, in the filter cover 5A formed by folding the one metal mesh material 7A, an outer edge 59A of the metal mesh material 7A is entirely included in the purification portion 50A in plan view. That is, in the state of the filter cover 5A being mounted to the filter body 3, the entire outer edge 59a of the filter cover 5A is included within the filter surface 32 in plan view.

As shown in the development view of FIG. 12, the metal mesh material 7A in the developed state has a long belt shape extending in left and right directions in FIG. 12.

The metal mesh material 7A in the developed state includes the central plane 70 having substantially the same rectangular shape as the filter surface 32, a first extension plane 71A extending outward from a first side 701 of the central plane 70, a second extension plane 72A extending outward from a second side 702 of the central plane 70, the first ear plane 73A extending outward from a third side 703 of the central plane 70, the second ear plane 74A extending outward from a fourth side 704 the central plane 70 in plan view.

The first extension plane 71A is demarcated into the first lower layer plane 715, a first inner side leg plane 716, a first outer side leg plane 717, and the first overhang plane 718A from outside to the central plane 70 side in that order by the three lines 711, 712, and 713A extending parallel to the first side 701. The first lower layer plane 715 has substantially the same rectangular shape as the central plane 70 in plan view, and constitutes a part of the purification portion 50A of the filter cover 5A in a folded state. The first inner side leg plane 716 has a belt shape extending along the first side 701 in plan view, and constitutes a part of the first leg portion 51A of the filter cover 5A in the folded state. The first outer side leg plane 717 has substantially the same belt shape as the first inner side leg plane 716 in plan view, and constitutes a part of the first leg portion 51A of the filter cover 5A in the folded state. The first overhang plane 718A has a belt shape extending along the first side 701 in plan view, and constitutes a part of the first overhang portion 57A of the filter cover 5A in the folded state. The first lower layer plane 715 and the first inner side leg plane 716 are demarcated by the line 711. The first inner side leg plane 716 and the first outer side leg plane 717 are demarcated by the line 712. The line 712 constitutes the first ridgeline 53A of the filter cover 5A in the folded state. The first outer side leg plane 717 and the first overhang plane 718A are demarcated by the line 713A.

The second extension plane 72A is demarcated into the second lower layer plane 725, a second inner side leg plane 726, a second outer side leg plane 727, and the second overhang plane 728A from outside to the central plane 70 side in that order by the three lines 721, 722, and 723A extending parallel to the second side 702. The second lower layer plane 725 has substantially the same rectangular shape as the central plane 70 in plan view, and constitutes a part of the purification portion 50A of the filter cover 5A in the folded state. The second inner side leg plane 726 has a belt shape extending along the second side 702 in plan view, and constitutes a part of the second leg portion 52A of the filter cover 5A in the folded state. The second outer side leg plane 727 has substantially the same belt shape as the second inner side leg plane 726 in plan view, and constitutes a part of the second leg portion 52A of the filter cover 5A in the folded state. The second overhang plane 728A has a belt shape extending along the second side 702 in plan view, and constitutes a part of the second overhang portion 58A of the filter cover 5A in the folded state. The second lower layer plane 725 and the second inner side leg plane 726 are demarcated by the line 721. The second inner side leg plane 726 and the second outer side leg plane 727 are demarcated by the line 722. The line 722 constitutes the second ridgeline 54A of the filter cover 5A in the folded state. The second outer side leg plane 727 and the second overhang plane 728A are demarcated by the line 723A.

The first ear plane 73A has a belt shape extending along the third side 703 in plan view and is smaller than the first and second extension planes 71A and 72A. The first ear plane 73A constitutes a part of the first edge part 55A of the filter cover 5A. The second ear plane 74A has a belt shape extending along the fourth side 704 in plan view and is smaller than the first and second extension planes 71A and 72A. The second ear plane 74A constitutes the second edge part 56A of the filter cover 5A in the folded state.

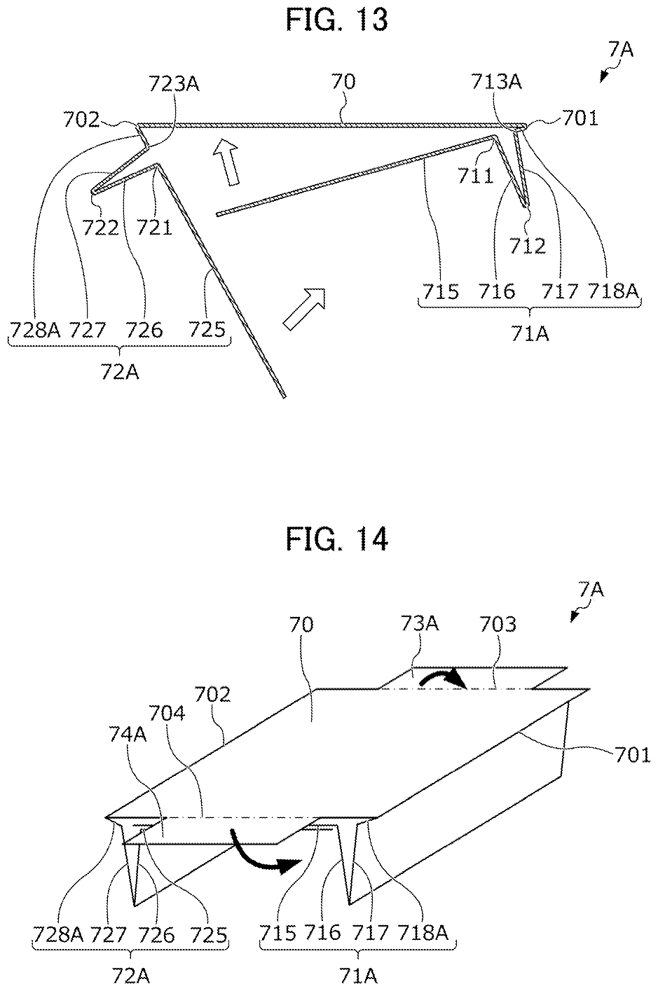

Each of FIGS. 13 and 14 is a diagram for illustrating a procedure for folding the metal mesh material 7A.

First, as shown in FIG. 13, after folding the first extension plane 71A along the lines 701, 711, 712, and 713A, the worker folds the second extension plane 72A along the lines 702, 721, 722, and 723A. More specifically, the worker folds the first extension plane 71A along the lines 701, 711, 712, and 713A so that the central plane 70 and the first lower layer plane 715 are stacked, the central plane 70 and the first overhang plane 718A are stacked, and the first inner side leg plane 716 and the first outer side leg plane 717 are stacked. Further, the worker folds the second extension plane 72A along the lines 702, 721, 722, and 723A so that the central plane 70 and the first and second lower layer planes 715 and 725 are stacked, the central plane 70 and the second overhang plane 728A are stacked, and the second inner side leg plane 726 and the second outer side leg plane 727 are stacked. Thereby, parts of the filter cover 5 to be the purification portion 50A, the first leg portion 51A, the second leg portion 52A, the first overhang portion 57A, and the second overhang portion 58A are formed.

Next, as shown in FIG. 14, after folding the first ear plane 73A along the third side 703, the worker further folds the second ear plane 74A along the fourth side 704. At this time, it is desirable to mountain-fold the third and fourth sides 703 and 704 when they are viewed from the central plane 70 side so that the first and second ear planes 73A and 74A are stacked on the second lower layer plane 725. Thereby, parts of the filter cover 5 to be the first and second edge parts 55A and 56A are formed.

By folding the metal mesh material 7A by the procedure as above, it is possible to include the entirety of an outer edge 78A, which is a part to become the outer edge 59A of the filter cover 5A, within the central plane 70.

Since a procedure for forming a minutely uneven surface having an antiviral action on the metal mesh material 7A folded by the procedure to manufacture the filter cover 5A as above, and a procedure for mounting the filter cover 5A to the filter body 3 to manufacture the filter device 2 are the same as those of the first embodiment, detailed description will be omitted.

According to the filter device 2A according to the present embodiment, the same effects as the filter device 2 according to the first embodiment are obtained.

Embodiments of the present invention have been described above. The present invention, however, is not limited thereto. Detailed configurations may be appropriately changed within the spirit of the present invention.

For example, though description has been made on the case where the purification portion 50/50A has a three-layer structure formed by folding the metal mesh material 7/7A so that three planes of the metal mesh material 7/7A are stacked in each of the above embodiments, the present invention is not limited thereto. The purification portion 50/50A may be in a multi-layer structure formed by folding the metal mesh material 7/7A so that two planes, or four or more planes of the metal mesh material 7/7A are stacked.

### EXPLANATION OF REFERENCE NUMERALS

1: Air conditioner
10: Air passage
2, 2A: Filter device
3: Filter body
30: Filter material
31: Frame member
32: Filter surface
5, 5A: Filter cover
50, 50A: Purification portion
51, 51A: First leg portion (leg portion)
52, 52A: Second leg portion (leg portion)
53, 53A: First ridgeline (ridgeline)
54, 54A: Second ridgeline (ridgeline)
59, 59A: Outer edge
7, 7A: Metal mesh material
70: Central plane
701: First side
702: Second side (second side)
703: Third side (third side)
704: Fourth side (fourth side)
71, 71A: First extension plane
711, 712, 713A: Line
715: First lower layer plane
72, 72A: Second extension plane
721, 722, 723A: Line
725: Second lower layer plane
73, 73A: First ear plane (third extension plane)
74, 74A: Second ear plane (fourth extension plane)
78, 78A: Outer edge

1. A filter device comprising:
a filter material; and
a mesh filter cover mounted to the filter material; wherein
the filter cover comprises a multi-layer purification portion formed by folding a metal mesh material; and
on the metal mesh material, a minutely uneven surface is formed.

2. The filter device according to claim 1, wherein
the uneven surface has an antiviral action.

3. The filter device according to claim 1, or wherein
the metal mesh material comprises a central plane having a rectangular shape in plan view, and extension planes extending outward from four sides of the central plane in a developed state; and
the filter cover is obtained by folding the metal mesh material on the four sides serving as fold lines.

4. The filter device according 1, wherein
the metal mesh material comprises a central plane having a rectangular shape in plan view, and first and second extension planes extending outwards from mutually facing first and second sides among four sides of the central plane, respectively, in a developed state; and
the filter cover is obtained by folding the metal mesh material on the first and second sides serving as fold lines so that the central plane and the first and second extension planes are stacked.

5. The filter device according to claim 4, wherein
the metal mesh material comprises third and fourth extension planes that extend outward from mutually facing third and fourth sides among the four sides in the developed state and are smaller than the first and second extension planes; and
the filter cover is obtained by folding the metal mesh material on the third and fourth sides serving as fold lines.

6. The filter device according to claim 1, wherein
the metal mesh material comprises a central plane having a rectangular shape in plan view, and an extension plane extending outward from one side of the central plane in a developed state;
the filter cover comprises the purification portion having a rectangular shape in plan view and including the central plane as one layer, and a leg portion intersecting with the purification portion and extending to the filter material side;
the leg portion is formed by folding the extension plane on a fold line parallel to the one side; and
a ridgeline of the leg portion on the filter material side is a mountain fold line when viewed from the filter material side.

7. The filter device according to claim 6, further comprising a frame member having a rectangular shape in plan view and holding an outer edge of the filter material; wherein
the leg portions are provided along an inner side of the frame member.

8. The filter device according to claim 6, further comprising a frame member having a rectangular shape in plan view and holding an outer edge of the filter material; wherein
the leg portions are provided along an outer side of the frame member.

9. A method for manufacturing a filter device, the filter device comprising a filter material and a filter cover mounted to the filter material, the method comprising:
folding a metal mesh material;
forming a minutely uneven surface on a surface of the metal mesh material that is folded, thereby manufacturing the filter cover; and
mounting the filter cover to the filter material.

10. An air conditioner comprising:
the filter device according to claim 1; and
an air passage through which air flows, wherein
the filter device is provided in the air passage; and
the filter cover is provided on an air upstream side with respect to the filter material.
